(19) United States
(12) Patent Application Publication
(10) Pub. No.: US 2019/0029974 A1
(43) Pub. Date: Jan. 31, 2019

(54) METHODS FOR TREATING SKIN DISORDERS AND CONDITIONS UTILIZING HAPTENS

(71) Applicant: RXi Pharmaceuticals Corporation, Marlborough, MA (US)

(72) Inventors: Gerard Cauwenbergh, Plainsboro, NJ (US); Keith Johnson, Durham, NC (US)

(73) Assignee: RXi Pharmaceuticals Corporation, Marlborough, MA (US)

(21) Appl. No.: 15/758,576

(22) PCT Filed: Sep. 9, 2016
(86) PCT No.: PCT/US16/51057

§ 371 Date: Mar. 8, 2018

Related U.S. Application Data
(60) Provisional application No. 62/217,683, filed on Sep. 11, 2015.
(60) Provisional application No. 62/262,871, filed on Dec. 3, 2015.

(51) Int. Cl. A61K 31/122; A61K 9/00; A61K 9/06; A61P 35/00; A61P 35/04; A61P 17/12

(52) U.S. Cl. CPC A61K 31/122; A61K 9/0014; A61P 17/12; A61P 35/00; A61P 35/04; A61K 9/06

(57) ABSTRACT

The invention pertains to the use of therapeutic approaches to treat various skin disorders and conditions. In particular, the invention pertains to the treatment of cutaneous cancers and cutaneous metastasis of solid tumors, as well as to the removal of tattoos, using a hapten. The invention provides various topical formulations comprising a hapten for treatment of these skin disorders and conditions.

### RELATED APPLICATIONS

This application claims the benefit under 35 U.S.C. § 119(e) of U.S. Provisional Application Ser. No. 62/217,683, entitled “METHODS FOR TREATING SKIN DISORDERS AND CONDITIONS UTILIZING HAPTENS,” filed on Sep. 11, 2015, and U.S. Provisional Application Ser. No. 62/262,871, entitled “METHODS FOR TREATING SKIN DISORDERS AND CONDITIONS UTILIZING HAPTENS,” filed on Dec. 3, 2015, the disclosure of each of which is herein incorporated by reference in its entirety.

### FIELD OF THE INVENTION

The invention pertains to the use of therapeutic approaches to treat various skin disorders and conditions. In particular, the invention pertains to the use of a hapten in the treatment of cutaneous cancers, cutaneous metastasis of solid tumors, cutaneous metastasis of malignant melanoma, and warts, as well as to the removal of tattoos. The invention provides various topical formulations comprising a hapten for treatment of these skin disorders and conditions.

### BACKGROUND

Haptens, such as Dinitrochlorobenzene (DNCB), Squaric Acid Dibutylester (SADBE) and Diphenylcyclopropenone (DPCP), are molecules that can bind to an endogenous protein to create a complete antigen which evokes contact hypersensitivity (CHS), which can clinically manifest as allergic contact dermatitis (ACD).

Cutaneous neoplasms, such as basal cell carcinoma and squamous cell carcinoma, are common cancers that usually present as discrete lesions on the skin. While they produce considerable morbidity by local invasion, they rarely metastasize. Conventional means of therapy, such as excision, electrodessication, curettage, radiotherapy, cryotherapy, and chemosurgery produce cure rates approaching 100%. However, in patients with certain skin disorders or conditions, such as basal cell nevus syndrome, xeroderma pigmentosum, arsenical dermatitis, extensive radiation dermatitis, and in fair-skinned subjects with severely actinically damaged skin, or in other cases in which the carcinomas are so numerous and/or widespread, conventional therapies become impractical.

Solid neoplasms often result in cutaneous metastasis. Cutaneous tissues generally do not receive an adequate amount of systemic anti-neoplastic drugs that are given by mouth or by parenteral route to treat the solid neoplasm. Thus, while the solid neoplasm is often adequately treated by such chemotherapeutic methods, the cutaneous metastasized lesions associated with such solid tumors are often more difficult to treat using these methods.

Tattoo removal is a common request in dermatologic surgery practices. Conventional tattoo removal methods include mechanical, chemical, and thermal methods, but these interventions may result in undesirable dermal damage, disfiguring scars, and pigmentary changes. While lasers are a commonly employed method of tattoo removal, numerous treatments are often needed and laser treatment may fail to eliminate the tattoo completely.

### SUMMARY OF THE INVENTION

In some aspects, the disclosure provides a method for treating cutaneous neoplasm, the method comprising administering to a subject in need thereof a therapeutically effective amount of a hapten that elicits a T-cell response.

In some embodiments, the cutaneous neoplasm is selected from the group consisting of basal cell carcinoma, squamous cell carcinoma, bowen's disease (pre-invasive squamous cell carcinoma), actinic keratosis, metastatic merkel cell carcinoma, and cutaneous T-cell lymphoma.

In some embodiments, the hapten is selected from the group consisting of diphenylcyclopropenone (DPCP), imiquimod, ingenol mebutate, and Squaric Acid Dibutylester (SADBE). In some embodiments, the hapten is DPCP.

In some aspects, the disclosure provides a method for the treatment of cutaneous metastasis in a subject having solid neoplasia, the method comprising administering to a subject in need thereof a therapeutically effective amount of a hapten that elicits a T-cell response.

In some embodiments, the hapten is administered as an adjunct or adjuvant therapy. In some embodiments, the subject is also receiving one or more anti-neoplastic therapies and/or one or more radiation therapies.

In some embodiments, the cutaneous metastasis originates from a solid neoplasia selected from the group consisting of breast carcinoma, lung carcinoma, bladder carcinoma, colon carcinoma, uterine carcinoma, renal carcinoma, gastrointestinal carcinoma, and oral squamous cell carcinoma. In some embodiments, the cutaneous metastasis is located in one or more cutaneous areas in a subject selected from the group consisting of the chest, abdomen, lower abdomen, back, scalp, head, neck, leg, arm, and other extremities, genitalia, pubic area, and pelvis areas.

In some embodiments, the hapten is selected from DPCP, imiquimod, ingenol mebutate, and SADBE. In some embodiments, the hapten is DPCP.

In some aspects, the disclosure provides a method for the complete or partial removal of a skin tattoo, the method comprising administering to a subject in need thereof a therapeutically effective amount of a hapten that elicits a T-cell response.

In some embodiments, the hapten is administered as an adjunct or adjuvant therapy. In some embodiments, the subject is also receiving laser treatment to completely or partially remove the tattoo. In some embodiments, the hapten is selected from DPCP, imiquimod, ingenol mebutate, and SADBE. In some embodiments, the hapten is DPCP.

In some embodiments, the hapten is administered to the skin of the subject as an initial sensitizing dose and a subsequent challenge dose. In some embodiments, the initial sensitizing dose and the subsequent challenge dose are administered to the same site on the skin. In some embodiments, the initial sensitizing dose is administered to a first site on the skin and the subsequent challenge dose is administered to second site on the skin that differs from the first site.

In some embodiments, the hapten is formulated in a composition comprising a gel formulation. In some embodiments, the composition comprises (a) a non-ionic surfactant, (b) an alcoholic ester, and (c) a gelling agent. In some embodiments, the non-ionic surfactant is selected from the group consisting of polyoxyethylene (20) monoleate, polyoxyethylene (20) sorbitan monooleate, polysorbate 80, palmitate, and stearate. In some embodiments, the alcoholic ester is selected from the group consisting of isopropyl myristate and isopropyl palmitate. In some embodiments, the gelling agent is selected from the group consisting of polyoxyl 40 stearate and hydroxypropyl cellulose. In some embodiments, the gel composition comprises polysorbate 80, isopropyl myristate, and polyoxyl 40 stearate.

In some embodiments, the gel composition comprises from about 0.1% to about 1% hapten. In some embodiments, the gel composition comprises 0.4% hapten. In some embodiments, the gel composition comprises from about 0.0000001% to about 0.4% hapten.

In some embodiments, the first challenge dose of hapten is administered two weeks or about two weeks subsequent to the administration of the sensitizing dose of hapten. In some embodiments, the challenge dose is administered to the skin daily. In some embodiments, the challenge dose is administered to the skin every other day. In some embodiments, the challenge dose is administered to the skin twice a week. In some embodiments, the challenge dose is administered to the skin weekly. In some embodiments, the challenge dose is administered to the skin every two weeks. In some embodiments, wherein the challenge dose is administered to the skin every three weeks. In some embodiments, the challenge dose is administered to the skin monthly. In some embodiments, the challenge dose is administered to the skin in any combination of daily, every other day, twice a week, weekly, every other week, every three weeks and/or monthly administration.

In some embodiments, the hapten is selected from DPCP, imiquimod, ingenol mebutate, and SADBE. In some embodiments, the hapten is DPCP.

In some aspects, the disclosure provides a composition for use in the treatment of a cutaneous neoplasm in a subject, wherein the composition comprises a hapten that elicits a T-cell response, a non-ionic surfactant, an alcoholic ester, and a gelling agent.

In some aspects, the disclosure provides a composition for use in the treatment of cutaneous metastasis in a subject having solid neoplasia, wherein the composition comprises a hapten that elicits a T-cell response, a non-ionic surfactant, an alcoholic ester, and a gelling agent.

In some aspects, the disclosure provides a composition for use in the complete or partial removal of a skin tattoo on a subject, wherein the composition comprises a hapten that elicits a T-cell response, a non-ionic surfactant, an alcoholic ester, and a gelling agent.

In some embodiments, the non-ionic surfactant is selected from the group consisting of polyoxyethylene (20) monoleate, polyoxyethylene (20) sorbitan monooleate, polysorbate 80, palmitate, and stearate. In some embodiments, the alcoholic ester is selected from is selected from the group consisting of isopropyl myristate and isopropyl palmitate. In some embodiments, the gelling agent is selected from the group consisting of polyoxyl 40 stearate and hydroxypropyl cellulose.

In some embodiments, the hapten is selected from DPCP, imiquimod, ingenol mebutate, and SADBE. In some embodiments, the hapten is DPCP. In some embodiments, the composition comprises DPCP, Polysorbate 80, Isopropyl myristate, and Polyoxyl 40 Stearate.

In some aspects, the disclosure provides a formulation for use in the treatment of a cutaneous neoplasm in a subject, the formulation comprising DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

In some aspects, the disclosure provides a formulation for use in the treatment of cutaneous metastasis in a subject having solid neoplasia, the formulation comprising DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

In some aspects, the disclosure provides a formulation for use in the complete or partial removal of a skin tattoo on a subject, the formulation comprising 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

In some embodiments of the formulations, the formulation comprises from about 0.1% to about 1% DPCP. In some embodiments, the formulation comprises 0.4% DPCP. In some embodiments, the formulation comprises from about 0.0000001% to about 0.4% DPCP.

In some aspects, the disclosure provides a method for the treatment of cutaneous neoplasm in a subject, the method comprising (a) administering to the skin of the subject a sensitizing dose of hapten; (b) administering to the skin of the subject a first challenge dose of hapten; and (c) continuing to administer to the skin of the subject one or more further challenge dose(s) of hapten according to a pre-determined schedule until the cutaneous neoplasm has been treated.

In some aspects, the disclosure provides a method for the treatment of cutaneous metastasis in a subject having solid neoplasia, the method comprising (a) administering to the skin of the subject a sensitizing dose of hapten; (b) administering to the skin of the subject a first challenge dose of hapten; and (c) continuing to administer to the skin of the subject one or more further challenge dose(s) of hapten according to a pre-determined schedule until the cutaneous metastasis has been treated.

In some aspects, the disclosure provides a method for the complete or partial removal of a tattoo in a subject, the method comprising (a) administering to the skin of the subject a sensitizing dose of hapten; (b) administering to the skin of the subject a first challenge dose of hapten; and (c) continuing to administer to the skin of the subject one or more further challenge dose(s) of hapten according to a pre-determined schedule until the tattoo has been completely or partially removed.

In some aspects, the disclosure provides a method for treating a wart, the method comprising administering to a subject in need thereof a therapeutically effective amount of a hapten that elicits a T-cell response.

In some aspects, the disclosure provides a method for treating cutaneous metastasis of malignant melanoma, the method comprising administering to a subject in need thereof a therapeutically effective amount of a hapten that elicits a T-cell response.

In some embodiments, the hapten is selected from the group consisting of diphenylcyclopropenone (DPCP), imiquimod, ingenol mebutate, and Squaric Acid Dibutylester (SADBE). In some embodiments, the hapten is DPCP.

In some embodiments, the hapten is formulated in a composition comprising a gel formulation. In some embodiments, the composition comprises (a) a non-ionic surfactant, (b) an alcoholic ester, and (c) a gelling agent. In some embodiments, the non-ionic surfactant is selected from the group consisting of polyoxyethylene (20) monoleate, polyoxyethylene (20) sorbitan monooleate, polysorbate 80, palmitate, and stearate. In some embodiments, the alcoholic ester is selected from the group consisting of isopropyl myristate and isopropyl palmitate. In some embodiments, the gelling agent is selected from the group consisting of polyoxyl 40 stearate and hydroxypropyl cellulose. In some embodiments, the composition comprises polysorbate 80, isopropyl myristate, and polyoxyl 40 stearate.

In some embodiments, the composition comprises from about 0.1% to about 1% hapten. In some embodiments, the composition comprises 0.4% hapten. In some embodiments, the composition comprises from about 0.0000001% to about 0.4% hapten.

In some aspects, the disclosure provides a composition for use in the treatment of a wart in a subject, wherein the composition comprises a hapten.

In some aspects, the disclosure provides a composition for use in the treatment of cutaneous metastasis of malignant melanoma in a subject, wherein the composition comprises a hapten.

In some embodiments, the hapten is selected from DPCP, imiquimod, ingenol mebutate, and SADBE. In some embodiments, the hapten is DPCP.

In some aspects, the disclosure provides a method for the treatment of a wart in a subject, the method comprising (a) administering to the skin of the subject a sensitizing dose of hapten; (b) administering to the skin of the subject a first challenge dose of hapten; and (c) continuing to administer to the skin of the subject one or more further challenge dose(s) of hapten according to a pre-determined schedule until the wart has been treated.

In some aspects, the disclosure provides a method for the treatment of cutaneous metastasis of malignant melanoma in a subject, the method comprising (a) administering to the skin of the subject a sensitizing dose of hapten; (b) administering to the skin of the subject a first challenge dose of hapten; and (c) continuing to administer to the skin of the subject one or more further challenge dose(s) of hapten according to a pre-determined schedule until the cutaneous metastasis of malignant melanoma has been treated.

In some embodiments, the hapten is formulated in a composition comprising a gel formulation or an ointment formulation.

In some aspects, the disclosure provides a composition comprising a hapten gel formulation, wherein the composition comprises a) a first co-solvent comprising a non-ionic surfactant, b) a second co-solvent comprising an alcoholic ester, and c) a gelling agent.

In some embodiments, the first co-solvent is selected from the group consisting of polyoxyethylene (20) monoleate, polyoxyethylene (20) sorbitan monooleate, polysorbate 80, palmitate and stearate, and wherein the second co-solvent is selected from the group consisting of isopropyl myristate and isopropyl palmitate, and wherein the gelling agent is selected from the group consisting of polyoxyl 40 stearate and hydroxypropyl cellulose. In some embodiments, the composition comprises 0.01 to 1% BHT, 10 to 20% Polysorbate 80, 10 to 20% Isopropyl myristate, 5 to 15% Propylene glycol, 0.1 to 5% Klucel and 40 to 70% Isopropyl alcohol. In some embodiments, the hapten is DPCP, imiquimod, ingenol mebutate or SADBE. In some embodiments, the hapten is DPCP.

In some aspects, the disclosure provides a composition comprising a hapten ointment formulation, wherein the composition comprises a) a first co-solvent comprising a non-ionic surfactant, b) a second co-solvent comprising an alcoholic ester, and c) a thickening agent.

In some embodiments, the first co-solvent is selected from the group consisting of polyoxyethylene (20) monoleate, polyoxyethylene (20) sorbitan monooleate, polysorbate 80, palmitate and stearate, wherein the second co-solvent is selected from the group consisting of isopropyl myristate and isopropyl palmitate, and wherein the thickening agent is selected from the group consisting of white wax, cetyl ester wax and glyceryl monosterate.

In some aspects, the disclosure provides a composition comprising a hapten ointment formulation, wherein the composition comprises 0.01 to 1% BHT, 20 to 50% Polysorbate 80, 20 to 50% Isopropyl myristate, 2.5 to 20% White wax, 2.5 to 20% Cetyl esters wax, 0 to 10% glyceryl monostearate, 0 to 1% methylparaben and/or 0 to 1% propylparaben.

In some embodiments, the hapten is DPCP, imiquimod, ingenol mebutate or SADBE. In some embodiments, the hapten is DPCP. In some embodiments, the dose of DPCP is 0.0000001% to about 1%.

In some aspects, the disclosure provides a method comprising administering to a subject in need thereof a therapeutically effective amount of a composition comprising a hapten gel formulation, wherein the composition comprises a) a first co-solvent comprising a non-ionic surfactant, b) a second co-solvent comprising an alcoholic ester, and c) a gelling agent.

In some embodiments, the first co-solvent is selected from the group consisting of polyoxyethylene (20) monoleate, polyoxyethylene (20) sorbitan monooleate, polysorbate 80, palmitate and stearate, and wherein the second co-solvent is selected from the group consisting of isopropyl myristate and isopropyl palmitate, and wherein the gelling agent is selected from the group consisting of polyoxyl 40 stearate and hydroxypropyl cellulose. In some embodiments, the gel composition is comprised of 0.01 to 1% BHT, 10 to 20% Polysorbate 80, 10 to 20% Isopropyl myristate, 5 to 15% Propylene glycol, 0.1 to 5% Klucel and 40 to 70% Isopropyl alcohol. In some embodiments, the hapten is DPCP, imiquimod, ingenol mebutate or SADBE. In some embodiments, the hapten is DPCP

In some aspects, the disclosure provides a method comprising administering to a subject in need thereof a therapeutically effective amount of a composition comprising a hapten ointment formulation, wherein the composition comprises a) a first co-solvent comprising a non-ionic surfactant, b) a second co-solvent comprising an alcoholic ester, and c) a thickening agent.

In some embodiments, the first co-solvent is selected from the group comprising polyoxyethylene (20) monoleate, polyoxyethylene (20) sorbitan monooleate, polysorbate 80, palmitate and stearate, and wherein the second co-solvent is selected from the group comprising of isopropyl myristate and isopropyl palmitate, and wherein the thickening agent is selected from the group comprising of white wax, cetyl ester wax and glyceryl monosterate.

In some aspects, the disclosure provides a method comprising administering to a subject in need thereof a therapeutically effective amount of a composition comprising a hapten ointment formulation, wherein the ointment is comprised of 0.01 to 1% BHT, 20 to 50% Polysorbate 80, 20 to 50% Isopropyl myristate, 2.5 to 20% White wax, 2.5 to 20% Cetyl esters wax, 0 to 10% glyceryl monostearate, 0 to 1% methylparaben and/or 0 to 1% propylparaben.

In some embodiments, the hapten is DPCP, imiquimod, ingenol mebutate or SADBE. In some embodiments, the hapten is DPCP. In some embodiments, the hapten is DPCP and wherein the dose of DPCP is about 0.0000001% to about 1%.

In some aspects, the disclosure provides a method comprising administering a therapeutically effective amount of a composition of claims 78-88 to a subject in need thereof.

In some embodiments, the method is a method for treating cutaneous neoplasm, cutaneous metastasis in a subject having solid neoplasia, cutaneous metastasis of malignant melanoma, or a wart, or wherein the method is a method for tattoo removal.

In some embodiments, a low sensitizing dose of a composition described herein is administered to a first site on the skin of a human patient followed by a subsequent administration to a second site on the skin of the patient a challenge dose of the composition, wherein the composition comprises DPCP. In some embodiments, the low sensitizing dose is about 0.1 to about 1% DPCP, and the challenge dose is 0.0000001% to about 0.4% DPCP. In some embodiments, the sensitizing dose is 0.4% DPCP.

In some embodiments, the challenge dose is administered to the skin daily. In other embodiments, the challenge dose is administered to the skin every other day. In some embodiments, the challenge dose is administered to the skin twice a week. In some embodiments, the challenge dose is administered to the skin weekly. In some embodiments, the challenge dose is administered to the skin every two weeks. In some embodiments, the challenge dose is administered to the skin three weeks. In some embodiments, the challenge dose is administered to the skin in any combination of daily, every other day, twice a week, weekly, every other week, every three weeks and/or monthly.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE FIGURES

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic graph showing the stability of DPCP in various solvents as determined by reverse phase HPLC.

FIG. 2 is a photograph showing the results of a gel formulation that has undergone rapid versus slow cooling.

FIG. 3 is a schematic graph showing a DPCP assay after 12 days at 50° C. The stability of DPCP in solvents was determined using reverse phase HPLC on a C18 column.

### DETAILED DESCRIPTION OF THE INVENTION

### Haptens

As used herein, the term “hapten” refers to a molecule that can bind to a protein, such as an endogenous protein, to create a complete antigen that evokes contact hypersensitivity (CHS). Non-limiting examples of haptens include Dinitrochlorobenzene (DNCB), Squaric Acid Dibutylester (SADBE), Diphenylcyclopropenone (DPCP), Imiquimod and Ingenol mebutate. CHS clinically manifests as allergic contact dermatitis (ACD). Without wishing to be bound by any theory, this may be achieved via the mechanism of delayed-type (Type IV) hypersensitivity (DTH). In DTH, the antigen that enters the skin (or the hapten-peptide complex formed after hapten entry) is captured by epidermal Langerhans cells or dermal dendritic cells.

This interaction begins a process of tethering, rolling, firm adhesion and diapedesis that culminates in extravasation of the T-cell; this cell is then guided to the antigen by chemokines produced by local skin cells, and in particular CTACK/CCL27, a skin-limited chemokine ligand to chemokine receptor CCR10 produced by basal keratinocytes and upregulated with cutaneous inflammation (see Levis et al., Topical immunotherapy of basal cell carcinomas with dinitrochlorobenzene; Cancer Res. 1973; 33:3036-42, herein incorporated by reference in its entirety). Initial exposure to the hapten produces an induction phase, which is generally subclinical; further contact (even with far lower doses) after up to ten days of effector T-cell expansion produces an elicitation phase, characterized by overt dermal inflammation (see Levis et al. Lymphokine production in cell mediated allergic dermatitis, Lancet 2: 389-390, 1973, herein incorporated by reference in its entirety).

Diphencyprone or Diphenylcyclopropenone (DPCP)

DPCP is a potent contact sensitizer that has distinct advantages for therapeutic use. The standard dose administered to a subject, 2.0% DPCP, is an overdose which causes the subject to become overly hypersensitized to the hapten during challenge. As a result of the sensitizing overdose, in earlier embodiments, the challenge doses had to be very low, ˜0.002% DPCP, due to hypersensitization. Recently, Levis et al. (US Patent Publication No. US 2011/0268761 A1, herein incorporated by reference in its entirety) demonstrated that a low sensitizing dose of about 0.4% DPCP gel compared to the standard sensitizing dose of 2.0% DPCP used in the art prevents the subject from becoming overly hypersensitive to the challenge dose. Lowering the sensitization dose allows for significantly higher challenge doses since the 0.4% sensitization dose does not overly hypersensitize the subject to the challenge dose. Also, a 0.4% sensitization dose allows for more frequent repeated application of the challenge dose (0.04%) which significantly enhances the immune response to DPCP. Avoidance of hyper-sensitization in patients to the challenge doses results in an improved safety and tolerability profile and a more robust therapeutic effect.

Imiquimod

Imiquimod is a small molecule immune response modifier that works through toll-like receptor 7 (TLR-7) to activate Langerhans cells, natural killer cells, macrophages and B-lymphocytes.

Ingenol Mebutate

Ingenol mebutate is a naturally isolated small molecule from the plant Euphorbia peplus used in the treatment of actinic keratoses.

Dinitrochlorobenzene (DNCB)

DNCB is a potent contact sensitizer that has been shown to stimulate the release of CD4+ helper T-cells and induce TH-1 type immunity by releasing cytokines, including Interleukin-2.

Squaric Acid Dibutylester (SADBE)

SADBE is a contact sensitizer that augments, stimulates, activates, potentiates, or modulates the immune response at either the cellular or humoral level. Its mode of action is either non-specific, resulting in increased immune responsiveness to a wide variety of antigens, or antigen-specific, i.e., affecting a restricted type of immune response to a narrow group of antigens. The therapeutic efficacy is related to its antigen-specific immunoadjuvanticity.

As used herein, the term “therapeutically effective amount” refers to an amount that provides a therapeutic or prophylactic benefit.

### Methods of Treatment

The present disclosure provides methods for the treatment of skin disorders and conditions, including various cutaneous neoplasms.

I. Methods for Treating Cutaneous Neoplasms

Haptens that elicit a T-cell response are efficacious for the treatment of cutaneous neoplasms. Exemplary T-cell responses may include cellular immune responses mediated by CD8+ T-cells, for example, capable of killing tumor and infected cells, and/or CD4+ T-cell responses, and/or humoral immune responses, for example, mediated by antibody-producing B-cells. In some embodiments, a T-cell response may include modulation of one or more gene expression markers of one or more T cell subsets (e.g., Th1, Th2, Th9, Th17, Th22, and/or regulatory T cells). For example, in one embodiment, a T-cell response includes upregulation of expression of one or more Th1-related genes such as IFNG and/or CXCL10.

In one aspect, the disclosure provides a method for treating cutaneous neoplasm comprising administering to a subject in need thereof a therapeutically effective amount of a hapten. In certain embodiments, the cutaneous neoplasm is selected from basal cell carcinoma, squamous cell carcinoma, bowen's disease (pre-invasive squamous cell carcinoma), actinic keratosis, metastatic merkel cell carcinoma, and cutaneous T-cell lymphoma. In one embodiment, the hapten elicits a T-cell response. In one embodiment, the hapten is selected from DPCP, imiquimod, ingenol mebutate, and SADBE. In one particular embodiment, the hapten is DPCP.

Basal Cell Carcinoma Nevus Syndrome

Basal cell carcinoma (BCC) (also known as Gorlins Syndrome) is an autosomal dominant disorder that results from mutations in the p53 and/or PTCH genes. Hahn et al., Cell 1996; 85(6):841-851; Johnson et al., Science 1996; 272 (5268):1668-1671. The PTCH gene is involved in the hedgehog signal transduction pathway and Gorlins syndrome.

BCC is the most common cancer in the United States, accounting for 75% of all skin cancers. With an incidence of 1 in 19,000, development of basal cell carcinomas is one of the major criteria for diagnosis and cause of morbidity and mortality. Evans et al., Am Journal of Medical Genetics A. 2010; 152:327-332. Basal carcinoma burden may vary from few to thousands of lesions and appear clinically and histopathologically similar to sporadic basal cell carcinomas, with similar rates of invasion and metastasis. Metastases are rare; tumors may locally invade surrounding tissues and bone.

Squamous Cell Carcinoma (SCC)

Squamous cell carcinoma (SCC) is an invasive malignant neoplasm of epidermal keratinocytes and is usually related to ultraviolet radiation exposure. In the United States, an estimated 700,000 cases of SCC are diagnosed every year.

Bowen's Disease

Bowen's Disease (also known as squamous cell carcinoma in situ and pre-invasive squamous cell carcinoma) is a rare neoplastic skin disease that is localized to the epidermis. The exact cause of Bowen's disease is unknown, however, chronic exposure to ultraviolet radiation and age are the two major risk factors for developing the disease.

Actinic Keratosis (AK)

Actinic Keratosus (AK) is pre-cancerous lesion that if left untreated, may turn into squamous cell carcinoma. Actinic keratosus is caused by chronic exposure to ultraviolet radiation. It is also referred to as solar keratosis and senile keratosis.

Metastatic Merkel Cell Carcinoma (MCC)

Metastatic Merkel Cell Carcinoma (MCC) is a rare aggressive neuroendocrine carcinoma located between the dermal-epidermal junction. MCC is caused by chronic exposure to ultraviolet radiation. In 2007, approximately 1,500 new cases of MCC were reported in the United States. MCC is also referred to as Toker tumor, primary small cell carcinoma of the skin, primary cutaneous neuroendocrine tumor and malignant trichodiscoma.

Cutaneous T-Cell Lymphoma (CTCL)

Cutaneous T-cell Lymphoma (CTCL) is a group of T-cell non-Hodgkin lymphomas that begins in the skin as an itchy, red rash that can thicken and form a tumor. CTCL is a group of lymphoproliferative disorders characterized by localization of neoplastic T lymphocytes to the skin. CTCL include the following: Mycosis fungiodes, Mycosis fungiodes variants and subtypes (folliculotropic mycosis fungoides, pagetoid reticulosis, granulomatous slack skin), Primary cutaneous CD30+ lymphoproliferative disorder, subcutaneous panniculitis-like T-cell Lymphoma, primary cutaneous CD4+ snakk/medium-sized pleomorphic T-cell lymphoma, Sezary syndrome, adult T-cell leukemia/lymphoma, extranodal NK/T-cell lymphoma (nasal type), primary cutaneous peripheral T-cell lymphoma, primary cutaneous aggressive epidermotropic CD8+ T-cell lymphoma and cutaneous gamma/delta positive T-cell lymphoma.

II. Methods for Treating Cutaneous Metastasis

Solid neoplasms often result in cutaneous metastasis. Cutaneous tissues generally do not receive an adequate amount of systemic anti-neoplastic drugs that are given by mouth or by parenteral route. Topical formulations of haptens, in conjunction with anti-neoplastic therapy, including chemotherapy and/or radiation treatment, would allow for the direct treatment of the cutaneous metastasis to treat the cutaneous lesions associated with solid tumors. Thus, in another aspect, the disclosure provides methods for the treatment of cutaneous metastasis, e.g., as an adjunct or adjuvant therapy, in cases where subjects have a solid tumor with cutaneous metastases.

Cutaneous metastatic carcinomas occurs in 3 to 10 percent of subjects with solid neoplasia. Cutaneous metastasis may occur in close proximity to the primary tumor, but may also occur at sites distant to the tumor. The main causes of cutaneous metastasis in women arise from breast cancer (23%), whereas the main causes of cutaneous metastasis in men results from lung and colon cancer. Typically cutaneous metastasis resulting from breast and lung carcinomas are located on the chest and neighboring areas. In addition, cutaneous metastasis of lung carcinomas may also be located on the abdomen, back and scalp. For oral squamous cell carcinomas, cutaneous metastasis are commonly found on the nearby skin of the head, neck and chest. Cutaneous metastasis of uterine carcinomas may be located on skin of genitalia and the pubic area, while cutaneous metastasis occurring from renal carcinoma may be located at distant sites, such as the skin of the head and neck. Cutaneous metastasis occurring from bladder carcinomas may be located at distant sites, including on the extremities, while cutaneous metastasis of gastrointestinal carcinomas may be located on the lower abdomen or pelvis. Clinical Atlas of Skin Tumors, 10 Jan. 2014, Cutaneous Metastasis, Baykal and Yazganoglu, chapter 16.

In one aspect, the disclosure provides a method for the treatment of cutaneous metastasis in a subject having solid neoplasia, the method comprising administering to a subject in need thereof a therapeutically effective amount of a hapten. In one embodiment, the hapten elicits a T-cell response. In certain embodiments, the hapten is selected from DPCP, imiquimod, ingenol mebutate, and SADBE. In one particular embodiment, the hapten is DPCP.

In certain embodiments of a method for treating cutaneous metastasis in a subject having solid neoplasia, the hapten is administered as an adjunct or adjuvant therapy. In some embodiments, the subject is also receiving one or more anti-neoplastic therapies. In some embodiments, the subject is receiving one or more radiation therapies. In some embodiments, the subject is receiving one or more anti-neoplastic therapies and/or one or more radiation therapies. In some embodiments, the subject is also receiving immunotherapy, e.g., but not limited to immune checkpoint inhibitor treatment such as an inhibitor that targets programmed death-1 (PD-1) receptor, e.g., pembrolizumab or nivolumab.

In certain embodiments of a method for the treatment of cutaneous metastasis in a subject having solid neoplasia, the cutaneous metastasis originates from a solid neoplasia selected from breast carcinoma, lung carcinoma, bladder carcinoma, colon carcinoma, uterine carcinoma, renal carcinoma, gastrointestinal carcinoma, and oral squamous cell carcinoma. In certain embodiments of a method for the treatment of cutaneous metastasis in a subject having solid neoplasia, the cutaneous metastasis is located in one or more cutaneous areas in a subject selected from the chest, abdomen, lower abdomen, back, scalp, head, neck, leg, arm, and other extremities, genitalia, pubic area, pelvis areas, and any other skin area in which cutaneous metastasis has occurred as a result of the presence of a solid neoplasia.

In some embodiments, hapten is administered as an adjunct or adjuvant therapy in conjunction with one or more anti-neoplastic therapies for the treatment of neoplasms selected from breast carcinoma, lung carcinoma, bladder carcinoma, colon carcinoma, uterine carcinoma, renal carcinoma, gastrointestinal carcinoma, and oral squamous cell carcinoma. Non-limiting examples of anti-neoplastic therapies for which hapten can serve as an adjunct or adjuvant therapy include, but are not limited to, cyclophosphamide (Cytoxan), docetaxel (Taxotere), doxorubicin (Adriamycin), epirubicin (Ellence), methotrexate (Maxtrex, Abitrexate, Folex, Folex PFS, Mexate, MexateAQ), paclitaxel (Taxol, Abraxane), 5-fluorouracil (Adrucil), carboplatin, trastuzumab (Herceptin), capecitabine (Xeloda), carboplatin (Paraplatin), cisplatin (Platinol, Platinol-AQ), eribulin (Halaven), gemcitabine hydrochloride (Gemzar), Ixabepilone (Ixempra), liposomal doxorubicin (Doxil), vinorelbine (Navelbine), tamoxifen (Nolvadex), anastrazole (Arimidex), letrozole (Femara), exemestane (Aromasin), goserelin (Zoladex), leuprolide (Lupron), fulvestrant (Faslodex), megestrol acetate (Megace), toremifene (Fareston), Abraxane (Paclitaxel Albumin-stabilized Nanoparticle Formulation), Afatinib Dimaleate, Alimta (Pemetrexed Disodium), Avastin (Bevacizumab), Carboplatin, Ceritinib, Crizotinib, Cyramza (Ramucirumab), Erlotinib Hydrochloride, Gefitinib, Gilotrif (Afatinib Dimaleate), Gemcitabine Hydrochloride, Iressa (Gefitinib), Mechlorethamine Hydrochloride, Mustargen (Mechlorethamine Hydrochloride), Navelbine (Vinorelbine Tartrate), Nivolumab, Opdivo (Nivolumab), Paclitaxel Albumin-stabilized Nanoparticle Formulation, Paraplat (Carboplatin), Pemetrexed Disodium, Ramucirumab, Tarceva (Erlotinib Hydrochloride), Vinorelbine Tartrate, Xalkori (Crizotinib), Zykadia (Ceritinib), Doxorubicin Hydrochloride, Etopophos (Etoposide Phosphate), Etoposide, Etoposide Phosphate, Hycamtin (Topotecan Hydrochloride), Toposar (Etoposide), VePesid (Etoposide), anastrozole (Arimidix), medroxyprogesterone acetate (Provera), Afinitor (Everolimus), Aldesleukin, Axitinib (Inlyta), Nexavar (Sorafenib Tosylate), Pazopanib Hydrochloride, Proleukin (Aldesleukin), Sorafenib Tosylate, Sunitinib Malate (Sutent), Temsirolimus (Torisel), Votrient (Pazopanib Hydrochloride), thiotepa, ramucirumab (Cyramza), fluorouracil (Efudex, Fluoroplex), Mitomycin C (Mitozytrex, Mutamycin), Lanreotide Acetate (Somatuline Depot), bleomycin (Blenoxane), Cetuximab, Erbitux (Cetuximab), and combination therapies thereof.

III. Methods for Treating Cutaneous Metastasis of Malignant Melanoma

As used herein, the term “skin cancer” refers to the malignant proliferation of any cells of the skin. Skin cancers include lentigo maligna, melanoma, keratoacanthoma, basal cell carcinoma (BCC), squamous cell carcinoma (SCC), Merkel cell carcinoma (MCC), sarcoma, angiosarcoma, cutaneous lymphoma, sweat gland carcinoma and sebaceous gland carcinoma, as disclosed in and incorporated by reference from US Patent Publication No. US 2015/0057362. As used herein, “melanoma” refers to a form of skin cancer that corresponds to a malignant proliferation of melanocytes. As disclosed in and incorporated by reference from US Patent Publication No. US 2015/0057362, in most melanomas, the initial phase, which occurs along the dermoepidermal junction and within the dermis, is referred to as the radial growth phase (RGP). Growth down through the epidermis, which occurs in the vertical growth phase (VGP), results in the malignant melanocytes contacting the lymphatic tissue and capillaries, which leads to metastasis.

Melanomas are the most aggressive form of skin cancer. Fortunately, most melanomas are discovered at an early stage when they are highly curable by surgery alone. According to data from the Surveillance, Epidemiology, and End Results Program (SEER), 84% of cutaneous melanomas are discovered while localized, and for these patients, the 5-year relative survival rate is 98%. The first line treatment for localized melanoma (Stage I and II) is surgical removal with adequate margins (Bichakjian 2011).

Once melanoma has spread beyond the localized area of the primary lesion, the survival rate decreases and melanoma becomes increasingly more difficult to treat successfully. Cases where metastases are present in the skin are a relatively rare occurrence. Cutaneous metastases can occur at two stages of melanoma which include Stage IIIc) ‘in transit’ metastases) and Stage IV (distant cutaneous metastases) (Balch 2009). In some cases the first sign of metastatic disease (Stage III) is the presence of ‘in-transit’ metastases where tumor nodules appear in the skin between the primary tumor and the closest draining lymph node.

When lesions are few and close together, surgery may still be an option; however, this represents a minority of cases. Once melanoma has spread beyond regionally through the lymph nodes, metastases can further be found in distal locations such as skin and internal organs (Leiter 2004).

Management of metastatic melanoma, including cutaneous metastases, is challenging and represents an area of great unmet need. Recently approved drugs offer some hope by adding time to survival but they have not been shown to greatly decrease the burden of the disease (Dunki-Jacobs 2013, Ott 2013).

Topical formulations of haptens, in conjunction with anti-neoplastic therapy, including chemotherapy and/or radiation treatment, would allow for the direct treatment of the cutaneous metastasis associated with malignant melanoma. Thus, in another aspect, the disclosure provides methods for the treatment of cutaneous metastasis, e.g., as an adjunct or adjuvant therapy, in cases where subjects have a melanoma with cutaneous metastases.

In one aspect, the disclosure provides a method for the treatment of cutaneous metastasis in a subject having malignant melanoma, the method comprising administering to a subject in need thereof a therapeutically effective amount of a hapten. In one embodiment, the hapten elicits a T-cell response. In certain embodiments, the hapten is selected from DPCP, imiquimod, ingenol mebutate, and SADBE. In one particular embodiment, the hapten is DPCP.

In certain embodiments of a method for treating cutaneous metastasis in a subject having malignant melanoma, the hapten is administered as an adjunct or adjuvant therapy. In some embodiments, the subject is also receiving one or more anti-neoplastic therapies. In some embodiments, the subject is receiving one or more radiation therapies. In some embodiments, the subject is receiving one or more anti-neoplastic therapies and/or one or more radiation therapies.

Non-limiting examples of anti-neoplastic therapies for which hapten can serve as an adjunct or adjuvant therapy include, but are not limited to, cyclophosphamide (Cytoxan), docetaxel (Taxotere), doxorubicin (Adriamycin), epirubicin (Ellence), methotrexate (Maxtrex, Abitrexate, Folex, Folex PFS, Mexate, MexateAQ), paclitaxel (Taxol, Abraxane), 5-fluorouracil (Adrucil), carboplatin, trastuzumab (Herceptin), capecitabine (Xeloda), carboplatin (Paraplatin), cisplatin (Platinol, Platinol-AQ), eribulin (Halaven), gemcitabine hydrochloride (Gemzar), Ixabepilone (Ixempra), liposomal doxorubicin (Doxil), vinorelbine (Navelbine), tamoxifen (Nolvadex), anastrazole (Arimidex), letrozole (Femara), exemestane (Aromasin), goserelin (Zoladex), leuprolide (Lupron), fulvestrant (Faslodex), megestrol acetate (Megace), toremifene (Fareston), Abraxane (Paclitaxel Albumin-stabilized Nanoparticle Formulation), Afatinib Dimaleate, Alimta (Pemetrexed Disodium), Avastin (Bevacizumab), Carboplatin, Ceritinib, Crizotinib, Cyramza (Ramucirumab), Erlotinib Hydrochloride, Gefitinib, Gilotrif (Afatinib Dimaleate), Gemcitabine Hydrochloride, Iressa (Gefitinib), Mechlorethamine Hydrochloride, Mustargen (Mechlorethamine Hydrochloride), Navelbine (Vinorelbine Tartrate), Nivolumab, Opdivo (Nivolumab), Paclitaxel Albumin-stabilized Nanoparticle Formulation, Paraplat (Carboplatin), Pemetrexed Disodium, Ramucirumab, Tarceva (Erlotinib Hydrochloride), Vinorelbine Tartrate, Xalkori (Crizotinib), Zykadia (Ceritinib), Doxorubicin Hydrochloride, Etopophos (Etoposide Phosphate), Etoposide, Etoposide Phosphate, Hycamtin (Topotecan Hydrochloride), Toposar (Etoposide), VePesid (Etoposide), anastrozole (Arimidix), medroxyprogesterone acetate (Provera), Afinitor (Everolimus), Aldesleukin, Axitinib (Inlyta), Nexavar (Sorafenib Tosylate), Pazopanib Hydrochloride, Proleukin (Aldesleukin), Sorafenib Tosylate, Sunitinib Malate (Sutent), Temsirolimus (Torisel), Votrient (Pazopanib Hydrochloride), thiotepa, ramucirumab (Cyramza), fluorouracil (Efudex, Fluoroplex), Mitomycin C (Mitozytrex, Mutamycin), Lanreotide Acetate (Somatuline Depot), bleomycin (Blenoxane), Cetuximab, Erbitux (Cetuximab), and combination therapies thereof.

IV. Methods for Treatment of Warts

Human papillomavirus (HPV), is a member of the papovavirus family, and comprises a non-enveloped small double-stranded DNA virus, which is capable of replicating in epithelial cells. HPV colonizes stratified epithelia. Non-limiting examples of stratified epithelia include skin, oral and genital mucosa. HPV can induce formation of benign tumors called papillomas (warts) or condylomas. As used herein, “wart” refers to benign epidermal tumors caused by HPV. More than 70 distinct HPV types have been identified. Each distinct HPV type has at least a 10% difference in the genome. HPV viruses are described further in and incorporated by reference from US Patent Publication No. US 2015/0057362.

Topical formulations of haptens would allow for the direct treatment of warts. Thus, in another aspect, the disclosure provides methods for the treatment of warts using a therapeutically effective amount of a hapten. In one aspect, the disclosure provides a method for the treatment of warts in a subject having a wart, the method comprising administering to a subject in need thereof a therapeutically effective amount of a hapten. In one embodiment, the hapten elicits a T-cell response. In certain embodiments, the hapten is selected from DPCP, imiquimod, ingenol mebutate, and SADBE. In one particular embodiment, the hapten is DPCP. In some embodiments, the subject is already receiving, or has already received, another treatment for warts.

V. Methods for Tattoo Removal

Tattoo removal is a common request in dermatologic surgery practices. Conventional tattoo removal methods include mechanical, chemical, and thermal methods, but these interventions may result in undesirable dermal damage, disfiguring scars, and pigmentary changes. Lasers are a commonly employed method of tattoo removal; however, numerous treatments are often needed and laser treatment may fail to eliminate the tattoo completely.

As discussed herein, certain haptens elicit a T-cell response to the skin. It has been demonstrated that tattoos can be removed by recruiting macrophages to the epidermal-dermal junction. Solis et al., Dermatologic Surgery, 2002, 28: 83-87; Ramirez et al., Dermatologic Surgery, 2007, 33: 319-325; U.S. Ser. No. 10/799,540. The macrophages breakdown the tattoo ink in the skin resulting in the removal of the tattoo. Haptens that work by eliciting a T-cell response to the areas of application therefore work as a topical tattoo remover.

Thus, in one aspect, the disclosure provides a method for the complete or partial removal of a tattoo, the method comprising administering to a subject in need thereof a therapeutically effective amount of a hapten. In one embodiment, the hapten elicits a T-cell response. In certain embodiments, the hapten is selected from DPCP, imiquimod, ingenol mebutate, and SADBE. In one particular embodiment, the hapten is DPCP.

In certain embodiments of a method for removing a tattoo on the skin of a subject, the hapten is administered as an adjunct or adjuvant therapy or treatment. Thus, in some embodiments, the subject is receiving one or more treatments (other than hapten treatment) for the complete or partial removal of a skin tattoo. In one embodiment, the subject is receiving laser treatment to completely or partially remove the tattoo. Lasers work by breaking up the tattoo ink in the skin into smaller particles that are more readily taken up by macrophages. Methods of laser tattoo removal include, but are not limited to continuous-wave lasers and Q-switched lasers. Thus, administration of a hapten can be used as an adjunct or adjuvant treatment to the removal of a tattoo by laser therapy, including continuous-wave laser and Q-switched laser therapy.

In some embodiments, administration of a hapten is used as an adjunct or adjuvant treatment to the removal of a tattoo by one or more other methods for removing tattoos, including, but not limited to, dermabrasion, trichloroacetic acid (TCA), salabrasion, cryosurgery, excision, intense pulsed light therapy (IPL), and cream removal products. Thus, administration of a hapten can be used as an adjunct or adjuvant treatment for the removal of a tattoo by one or more therapies selected from dermabrasion, trichloroacetic acid (TCA), salabrasion, cryosurgery, excision, intense pulsed light therapy (IPL), and cream removal products. In some embodiments, tattoo removal consists of 1, 2, 3, 4, 5, or more removal treatments before the tattoo is fully removed. Adjunct or adjuvant treatment with a hapten, such as DPCP, following or in conjunction with another tattoo removal treatment can increase the rate and success of tattoo removal.

### Compositions

The disclosure provides compositions comprising one or more haptens that are useful in the treatment of cutaneous neoplasms, cutaneous metastasis, warts, and tattoo removal. Thus, in one aspect, the present disclosure provides compositions comprising a hapten. In some embodiments, the hapten elicits a T-cell response. In some embodiments, the hapten is selected from diphenylcyclopropenone (DPCP), imiquimod, ingenol mebutate, and Squaric Acid Dibutylester (SADBE). In certain particular embodiments, the hapten is DPCP.

In some embodiments, the hapten composition is formulated as a gel. In some embodiments, the composition comprises a hapten, a non-ionic surfactant, an alcoholic ester, and optionally a gelling agent. In some embodiments, the hapten is formulated in a gel composition. In some embodiments, the gel composition comprises (a) a non-ionic surfactant, (b) an alcoholic ester, and (c) a gelling agent. In some embodiments, the non-ionic surfactant is selected from polyoxyethylene (20) monoleate, polyoxyethylene (20) sorbitan monooleate, polysorbate 80, palmitate, and stearate. In certain particular embodiments, the non-ionic surfactant is polysorbate 80. In some embodiments, the alcoholic ester is selected from isopropyl myristate and isopropyl palmitate. In certain particular embodiments, the alcoholic ester is isopropyl myristate. In some embodiments, the gelling agent is polyoxyl 40 stearate. Thus, in some embodiments, the composition comprises a hapten, a non-ionic surfactant selected from polyoxyethylene (20) monoleate, polyoxyethylene (20) sorbitan monooleate, polysorbate 80, palmitate and stearate; an alcoholic ester selected from isopropyl myristate and isopropyl palmitate; and a gelling agent that is polyoxyl 40 stearate. In certain embodiments, the composition comprises a hapten, polysorbate 80, isopropyl myristate, and polyoxyl 40 stearate. In one particular embodiment, the composition is a formulation comprising DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

In some embodiments, a composition comprising a hapten, such as DPCP, is formulated as a gel comprising a) a first co-solvent comprising a non-ionic surfactant, b) a second co-solvent comprising an alcoholic ester, and c) a gelling agent. The first co-solvent is selected from the group consisting of polyoxyethylene (20) monoleate, polyoxyethylene (20) sorbitan monooleate, palmitate and stearate, and wherein the second co-solvent is selected from the group consisting of isopropyl myristate and isopropyl palmitate, and wherein said gelling agent is polyoxyl 40 stearate.

Alternatively, the gel can be comprised of a) a first co-solvent comprising a non-ionic surfactant, b) a second co-solvent comprising an alcoholic ester, c) an alcohol and d) a thickening agent. The first co-solvent is selected from the group consisting of polyoxyethylene (20) monoleate, polyoxyethylene (20) sorbitan monooleate, polysorbate 80 (PS80), palmitate and stearate, and wherein the second co-solvent is selected from the group consisting of isopropyl myristate and isopropyl palmitate, wherein the alcohol is selected from the group of ethanol or isopropanol and wherein the gelling agent is hydroxypropyl cellulose (Klucel™).

In other embodiments, a composition comprising a hapten, such as DPCP, is formulated as an ointment. The ointment can comprise a) a first co-solvent comprising a non-ionic surfactant, b) a second co-solvent comprising an alcoholic ester, and c) a thickening agent. The first co-solvent is selected from the group consisting of polyoxyethylene (20) monoleate, polyoxyethylene (20) sorbitan monooleate, palmitate and stearate, and wherein the second co-solvent is selected from the group consisting of isopropyl myristate and isopropyl palmitate, and wherein the thickening agent is selected from the group of and/or any combination of white wax, cetyl ester wax and/or glyceryl monostearate.

In other embodiments, compositions comprising one or more haptens, such as DPCP, can be formulated as a cream, lotion, foam, patch or paste.

The compositions described herein can contain one or more haptens at any therapeutically effective amount. In some embodiments, the composition may comprise a sensitizing dose of hapten. In certain of the embodiments described herein, the composition comprises from about 0.1% to about 1% hapten. In some embodiments, the composition comprises at least 0.1%, at least 0.2%, at least 0.3%, at least 0.4%, at least 0.5%, at least 0.6%, at least 0.7%, at least 0.8%, at least 0.9% or at least 1% hapten. In certain particular embodiments, the composition comprises 0.4% hapten. In other embodiments, the composition may comprise a challenge dose, e.g., as described herein. In certain of the embodiments described herein, the composition comprises from about 0.0000001% to about 0.4% hapten. In some embodiments the hapten is selected from diphenylcyclopropenone (DPCP), imiquimod, ingenol mebutate, and Squaric Acid Dibutylester (SADBE). In certain particular embodiments, the hapten is DPCP.

In some embodiments, the gel formulation containing a hapten can comprise one or more of the following excipients: BHT, Klucel MF Pharm, isopropyl alcohol, propylene glycol, polysorbate 80, and/or isopropyl myristate. In some embodiments, the percentages w/w of these excipients correspond to approximately 0.1%, 2%, 57.9%, 10%, 15%, and 15%, respectively. In some embodiments, the excipients are reduced slightly in formulations containing DPCP.

In some embodiments, the ointment formulation containing a hapten can comprise one or more of the following excipients: BHT, methylparaben, propylparaben, cetyl esters wax, white wax, polysorbate 80, and isopropyl myristate. In some embodiments, the percentages w/w of these excipients corresponds to approximately 0.1%, 0.1%, 0.05%, 10%, 10%, 39.875%, and 39.875%, respectively. In some embodiments, the excipients are reduced slightly in formulations containing DPCP.

In some embodiments, the ointment formulation containing a hapten can comprise one or more of the following excipients: BHT, methylparaben, propylparaben, glyceryl monostearate, EP, cetyl esters wax, white wax, polysorbate 80, and isopropyl myristate. In some embodiments, the percentages w/w of these excipients correspond to 0.1%, 0.1%, 0.05%, 5%, 7.5%, 7.5%, 39.875%, and 39.875%, respectively. In some embodiments, the excipients are reduced slightly in formulations containing DPCP.

### Administration

The present disclosure provides methods for treating various skin disorders and conditions by administering a hapten that elicits a T-cell response. Without wishing to be bound by any theory, the immune response induced in a subject by administering a hapten, such as DPCP, may include cellular immune responses mediated by CD8+ T-cells capable of killing tumor and infected cells, and CD4+ T-cell responses. Humoral immune responses, mediated primarily by antibody-producing B-cells may also be induced.

In some embodiments, the disclosure provides methods for sensitizing a subject to a therapeutic modality by administering an initial sensitizing dose of hapten to a subject followed by a subsequent administration of challenge dose of hapten to the subject. Thus, in some embodiments, to enhance an immune response in a subject, the hapten is administered to the skin of a subject in an initial sensitizing dose (which elicits sensitivity to subsequent treatment) and one or more subsequent challenge dose(s).

In some embodiments, the disclosure provides a method for the treatment of cutaneous neoplasm in a subject, the method comprising (a) administering to the skin of a subject a sensitizing dose of hapten; (b) administering to the skin of the subject a first challenge dose of hapten; and (c) continuing to administer to the skin of the subject one or more further challenge dose(s) of hapten according to a pre-determined schedule until the cutaneous neoplasm has been treated.

In some embodiments, the disclosure provides a method for the treatment of cutaneous metastasis in a subject having solid neoplasia, the method comprising (a) administering to the skin of a subject a sensitizing dose of hapten; (b) administering to the skin of the subject a first challenge dose of hapten; and (c) continuing to administer to the skin of the subject one or more further challenge dose(s) of hapten according to a pre-determined schedule until the cutaneous metastasis has been treated.

In some embodiments, the disclosure provides a method for the treatment of cutaneous metastasis of malignant melanoma, the method comprising (a) administering to the skin of a subject a sensitizing dose of hapten; (b) administering to the skin of the subject a first challenge dose of hapten; and (c) continuing to administer to the skin of the subject one or more further challenge dose(s) of hapten according to a pre-determined schedule until the cutaneous metastasis has been treated.

In some embodiments, the disclosure provides a method for the treatment of a wart in a subject having a wart, the method comprising (a) administering to the skin of a subject a sensitizing dose of hapten; (b) administering to the skin of the subject a first challenge dose of hapten; and (c) continuing to administer to the skin of the subject one or more further challenge dose(s) of hapten according to a pre-determined schedule until the wart has been treated.

In some embodiments, the disclosure provides a method for the complete or partial removal of a tattoo in a subject, the method comprising (a) administering to the skin of a subject a sensitizing dose of hapten; (b) administering to the skin of the subject a first challenge dose of hapten; and (c) continuing to administer to the skin of the subject one or more further challenge dose(s) of hapten according to a pre-determined schedule until the tattoo has been completely or partially removed.

In any of these embodiments, the sensitizing dose of the hapten can range from 0.1% to 1% hapten, including approximately 0.1%, 0.2%, 0.3%, 0.4%, 0.5%, 0.6%, 0.7%, 0.8%, 0.9%, and 1.0% hapten. In certain particular embodiments, the sensitizing dose of the hapten is 0.4% or about 0.4% hapten. In any of these embodiments, the challenge dose of the hapten can range from 0.0000001% to 0.4% hapten (any integer between and including 0.0000001 and 0.4). For example, the challenge dose of the hapten can range from 0.01%-0.2% hapten, or 0.01%-0.04% hapten, or 0.04%-0.4% hapten, or 0.04%-0.2% hapten. In any of these embodiments, the hapten can be selected from DPCP, imiquimod, ingenol mebutate, and SADBE. In certain particular embodiments, the hapten is DPCP.

In any of these embodiments, the sensitizing dose of hapten can be administered to the skin two weeks or approximately two weeks prior to the administration of the first challenge dose of hapten. In any of these embodiments, the first challenge dose can be administered to the skin subsequent to the sensitizing dose. In some embodiments, the first challenge dose is administered to the skin two weeks or about two weeks after the sensitizing dose. In some embodiments, the first challenge dose is administered to the skin earlier or later than two weeks after the sensitizing dose. For example, the first challenge dose can be administered from about 1-25 days following the initial sensitizing dose, including 1, 2, 3, 4, 5, 6, 7, 8, 9, 10, 11, 12, 13, 14, 15, 16, 17, 18, 19, 20, 21, 22, 23, 24, or 25 days following the sensitizing dose. In any of these embodiments, the first challenge dose of hapten can be administered to the skin following the sensitization dose and then subsequently administered on a schedule selected from 1-5 times daily, twice a day, once a day, every other day, twice a week, once a week, once every two weeks, once every three weeks, once a month, once every two months or longer, and in any schedule combination thereof until the skin disorder or condition is treated (e.g., cutaneous neoplasm, cutaneous metastasis is treated, wart is treated, or the skin tattoo is completely or partially removed). In the case of a relapse or insufficient or incomplete therapeutic effect, dosing can be re-initiated.

In any of these embodiments, the first challenge dose and the subsequent continuing challenge doses can be the same dose. In other embodiments, the first challenge dose and the subsequent continuing challenge doses can be different doses. For example, in some embodiments, alternating high and low challenge doses can be administered. In one embodiment, alternating 0.4% and 0.04% concentrations of a hapten can be administered. In any of the embodiments disclosed herein, the sensitizing dose and the challenge dose(s) of hapten can be administered to the same site on the skin. In any of the embodiments described herein, the sensitizing dose and the challenge dose(s) can be administered to different sites on the skin. For example, the sensitizing dose may be applied to a normal skin area and the challenge dose may be applied to the tumor, metastasis site, wart or tattoo site. Thus, in any of these embodiments, the sensitizing and/or the challenge dose(s) may be administered to the neoplastic or tumor site or to normal skin. Common skin sites for administration of sensitizing dose include the upper arm or leg area and lymph node areas. It should be appreciated that dosing of a hapten could be optimized by one of ordinary skill in the art without undue experimentation.

In any of these embodiments, the hapten can be formulated in any of the compositions discussed above, including gel or ointment formulations. In some embodiments, the composition comprises a non-ionic surfactant selected from polyoxyethylene (20) monoleate, polyoxyethylene (20) sorbitan monooleate, polysorbate 80, palmitate and stearate; an alcoholic ester selected from isopropyl myristate and isopropyl palmitate; and a gelling agent that is polyoxyl 40 stearate. In certain embodiments, the composition comprises a hapten, polysorbate 80, isopropyl myristate, and polyoxyl 40 stearate. In one particular embodiment, the composition is a formulation comprising DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

In some embodiments, a composition comprising a hapten, such as DPCP, is formulated as a gel comprising a) a first co-solvent comprising a non-ionic surfactant, b) a second co-solvent comprising an alcoholic ester, and c) a gelling agent. The first co-solvent can be selected from the group consisting of polyoxyethylene (20) monoleate, polyoxyethylene (20) sorbitan monooleate, palmitate and stearate, wherein the second co-solvent can be selected from the group consisting of isopropyl myristate and isopropyl palmitate, and wherein said gelling agent is polyoxyl 40 stearate.

Alternatively, the gel can be comprised of a) a first co-solvent comprising a non-ionic surfactant, b) a second co-solvent comprising an alcoholic ester, c) an alcohol and d) a thickening agent. The first co-solvent can be selected from the group consisting of polyoxyethylene (20) monoleate, polyoxyethylene (20) sorbitan monooleate, polysorbate 80 (PS80), palmitate and stearate, wherein the second co-solvent can be selected from the group consisting of isopropyl myristate and isopropyl palmitate, wherein the alcohol can be selected from the group of ethanol or isopropanol and wherein the gelling agent is hydroxypropyl cellulose (Klucel™).

In other embodiments, a composition comprising a hapten, such as DPCP, is formulated as an ointment. The ointment can comprise a) a first co-solvent comprising a non-ionic surfactant, b) a second co-solvent comprising an alcoholic ester, and c) a thickening agent. The first co-solvent can be selected from the group consisting of polyoxyethylene (20) monoleate, polyoxyethylene (20) sorbitan monooleate, palmitate and stearate, and wherein the second co-solvent can be selected from the group consisting of isopropyl myristate and isopropyl palmitate, and wherein the thickening agent can be selected from the group of and/or any combination of white wax, cetyl ester wax and/or glyceryl monostearate.

In other embodiments, compositions comprising one or more haptens, such as DPCP, can be formulated as a cream, lotion, foam, patch or paste.

Hapten compositions may be applied to the skin by dabbing a cotton-tipped swab that has been saturated with solution onto the skin at the desired site of application, without repeated rubbing or spreading of the solution over an extended area. For both the sensitization and treatment applications, the hapten composition is preferably left on the skin for a period of time before washing it off. In some embodiments, the hapten composition is left on the skin for a time period selected from about 1-72 hours, about 2-60 hours, about 3-48 hours, about 4-36 hours, and about 8-24 hours.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1: Compatibility of DPCP with Solvents for Gel and Ointment Formulations

The compatibility and solubility of DPCP was determined in both isopropyl myristate (IPM) as well as Polysorbate 80 (PS80). The solubility of DPCP in IPM is about 1.1% w/w and DPCP was found to be highly soluble in PS80. Next, the stability of DPCP in these solvents was determined. A solution of 0.4% DPCP in isopropyl myristate and a solution of 0.4% DPCP in Polysorbate 80 was placed at 50° C. for two weeks. The stability of DPCP in these solvents was determined using reverse phase HPLC on a C18 column.

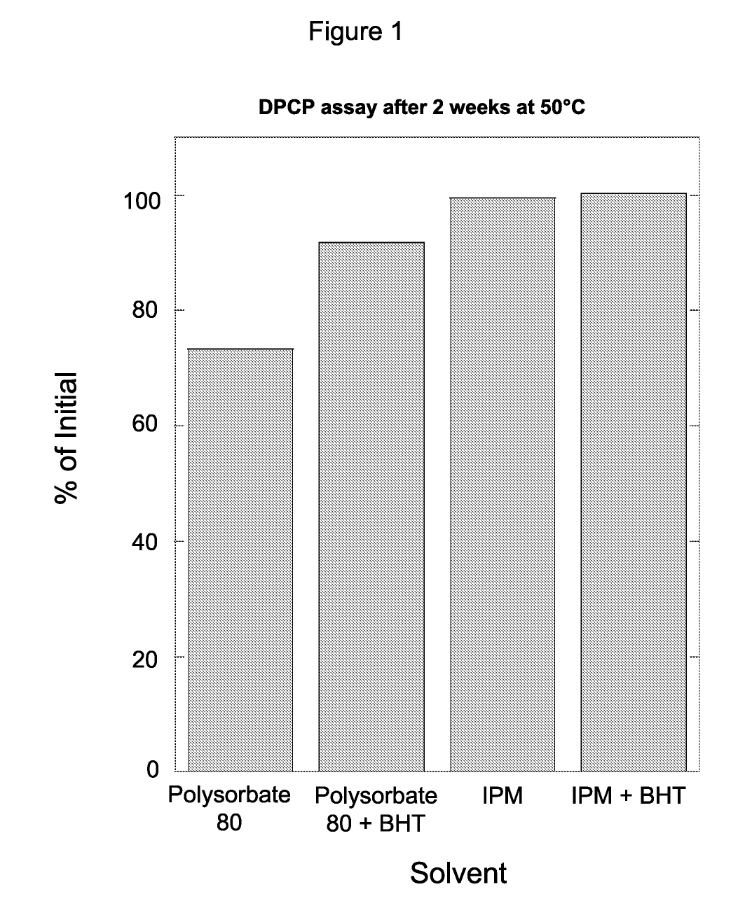

DPCP is stable in IPM at accelerated conditions; however, some degradation of DPCP was observed in the presence of PS80 (FIG. 1). Butylated hydroxytoloune (BHT) was shown to reduce the amount of degradation of DPCP in PS80.

### Example 2: The Effect of the Rate of Cooling on the Structure of Gel Formulation

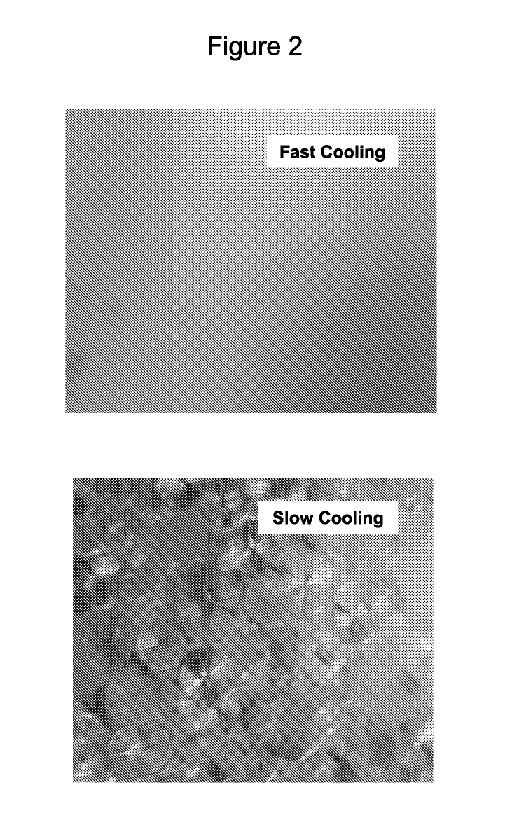

The effect of the rate of cooling on the structure of the following gel formulation was tested: 0.02% Butylated hydroxytoloune (BHT), 43.915% Polysorbate 80, 43.195% Isopropyl myristate, 12% Polyoxyl 40 Stearate, 0.1% Methyl Paraben and 0.05% Propyl Paraben. The rate of cooling was varied during the manufacture of said formulation to determine the effect on the formulation's physical properties. Two cooling rates were tested, the first was rapid cooling on ice, the other was a slow cooling, where the temperature of the vessel containing the formulation was reduced by about 5° C. every 10 to 15 minutes. The formulations were observed using a microscope with cross polarized light (160× images) to determine the physical structure of the formulation (FIG. 2). Utilizing the slow cooling method, a significantly more structured phase was observed compared to fast cooling. In addition, with fast cooling, the formulation appears mostly amorphous with some small areas with crystalline structure.

### Example 3: Use of DPCP Formulation in the Treatment of Cutaneous Neoplasms

Adult human subjects between the ages of 18-75 having 25 or more basal cell carcinomas are treated with a control formulation (no DPCP) or a DPCP formulation comprising 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben. All subject tumors are recorded noting their location, size, and characteristics and photographed prior to treatment. Representative tumors over 5 mm are biopsied. 0.1 ml of a sensitizing dose of 0.4% DPCP is applied to the skin of the subjects (upper arm area), allowed to dry, and covered with an adhesive bandage (washed off in 24 hours). The control subjects receive formulation with no DPCP. Two weeks later, a challenge dose of 0.01-0.04% DPCP is administered to the tumor site(s). Tumors are treated once daily with the challenge dose for a period of eight weeks. Following treatment, patients are assessed and tumors are examined and compared to their size and characteristics prior to treatment to determine efficacy. Efficacy for each target tumor is graded and any adverse reactions are reviewed, recorded, and treated as medically necessary. Further courses of treatment for additional eight week periods are administered if the tumor(s) fail to respond or respond poorly.

### Example 4: Use of DPCP Formulation as an Adjuvant Therapy in the Treatment of Cutaneous Metastasis

Subjects with breast cancer undergoing chemotherapy and/or radiation are treated with a control formulation (no DPCP) or a DPCP formulation comprising 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben. Metastasis is recorded, noting location, size, and characteristics of metastasized area, and photographed prior to treatment. A sensitizing dose of 0.4% DPCP is applied to the skin of the subjects (lymph node area), allowed to dry, and covered with an adhesive bandage. The control subjects receive formulation with no DPCP. Two weeks later, a challenge dose of 0.04%-0.2% DPCP is administered to the site of metastasis. The metastasis site is treated twice a week with the challenge dose for a period of eight weeks. Following treatment, tumors are examined and compared to their size and characteristics prior to treatment to determine efficacy. Efficacy for each site is graded and any adverse reactions are reviewed, recorded, and treated as medically necessary. Further courses of treatment for additional eight week periods are administered if the tumor(s) fail to respond or respond poorly.

### Example 5: Use of DPCP Formulation in the Treatment of Cutaneous Metastasis of Malignant Melanoma

Adult human subjects between the ages of 18-75 having one or more symptoms of cutaneous metastasis of malignant melanoma are treated with approximately a control formulation (no DPCP) or a DPCP formulation comprising 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben. 0.1 ml of a sensitizing dose of 0.4% DPCP is applied to the skin of the subjects (upper arm area), allowed to dry, and covered with an adhesive bandage (washed off in 24 hours). The control subjects receive formulation with no DPCP. Two weeks later, a challenge dose of 0.01-0.04% DPCP is administered to the tumor site(s). Cutaneous metastases are treated once daily with the challenge dose for a period of eight weeks. Following treatment, patients are assessed to determine efficacy. Efficacy for each cutaneous metastasis is graded and any adverse reactions are reviewed, recorded, and treated as medically necessary. Further courses of treatment for additional eight week periods are administered if the cutaneous metastasis fail to respond or respond poorly.

### Example 6: Use of DPCP Formulation in the Treatment of Warts

Adult human subjects between the ages of 18-75, who have a wart, are treated with a control formulation (no DPCP) or a DPCP formulation comprising 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben. All warts are recorded noting their location, size, and characteristics and photographed prior to treatment. 0.1 ml of a sensitizing dose of 0.4% DPCP is applied to the skin of the subjects (wart area), allowed to dry, and covered with an adhesive bandage (washed off in 24 hours). The control subjects receive formulation with no DPCP. Two weeks later, a challenge dose of 0.01-0.04% DPCP is administered to the tumor site(s). Warts are treated once daily with the challenge dose for a period of eight weeks. Following treatment, patients are assessed and warts are examined and compared to their size and characteristics prior to treatment to determine efficacy. Efficacy for each target wart is graded and any adverse reactions are reviewed, recorded, and treated as medically necessary. Further courses of treatment for additional eight week periods are administered if the warts fail to respond or respond poorly.

### Example 7: Use of DPCP Formulation in the Removal of Tattoos

The efficacy of a topical formulation of DPCP for the removal of tattoos in a guinea pig model is evaluated. Albino guinea pigs are tattooed with black, red, green, and yellow pigment. After tattooing, the subject guinea pigs either receive no treatment (control), are treated with the topical formulation containing no DPCP (control), or are treated with the topical formulation containing DPCP. The formulation comprises 0.4% DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben. The animals are treated daily for 7 days. Biopsies of the tattoos are taken at 6 hours, 7 days, and 28 days. Visualization of the tattoos in the various subject groups at these time points indicate whether the formulation has worked to completely or partially remove the tattoo.

In another study, the same methods and formulations are used except that a 0.4% DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben sensitizing formulation is applied to the tattoo area. Two weeks later, a challenge formulation comprising 0.04% DPCP, 0.02% Butylated hydroxytoloune (BHT), 43.4125 to 43.915% Polysorbate 80, 43.4125 to 43.915% Isopropyl myristate, 12% Polyoxyl 40 Stearate, 0.1% Methyl Paraben and 0.05% Propyl Paraben is applied to the tattoo area daily for 7 days. Biopsies of the tattoos are taken at 6 hours (after first challenge dose), 7 days, and 28 days. Visualization of the tattoos in the various subject groups at these time points indicate whether the formulation has worked to completely or partially remove the tattoo.

### Example 8: Use of a DPCP Formulation as an Adjuvant Therapy in the Removal of Tattoos

The efficacy of topical DPCP formulation is evaluated as an adjuvant to laser removal of mature tattoos. Albino guinea pigs are tattooed with black ink, then randomly assigned into two groups: one group undergoes sequential laser treatments with a Q-switched alexandrite laser in conjunction with biweekly applications of 4% DPCP gel formulation, while the other group undergoes laser therapy alone. The DPCP formulation comprises: 0.4% DPCP, 0.02% Butylated hydroxytoloune (BHT), 43.4125 to 43.915% Polysorbate 80, 43.4125 to 43.915% Isopropyl myristate, 12% Polyoxyl 40 Stearate, 0.1% Methyl Paraben and 0.05% Propyl Paraben. Subjects are evaluated with clinical photographs and skin biopsies after six laser treatment sessions to determine whether topical DPCP formulation is a useful adjuvant to laser tattoo removal.

In another study, the same methods and formulations are used except that a 0.4% DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben sensitizing formulation is applied to the tattoo area two weeks prior to administration of a challenge formulation comprising 0.04% DPCP, 0.02% Butylated hydroxytoloune (BHT), 43.4125 to 43.915% Polysorbate 80, 43.4125 to 43.915% Isopropyl myristate, 12% Polyoxyl 40 Stearate, 0.1% Methyl Paraben and 0.05% Propyl Paraben. The challenge formulation is applied to the tattoo area twice daily for 7 days. Biopsies of the tattoos are taken at 6 hours (after first challenge dose), 7 days, and 28 days. Visualization of the tattoos in the various subject groups at these time points indicate whether the formulation has worked to completely or partially remove the tattoo.

### Example 9: Stability of DPCP in Ethanol and Isopropyl Alcohol

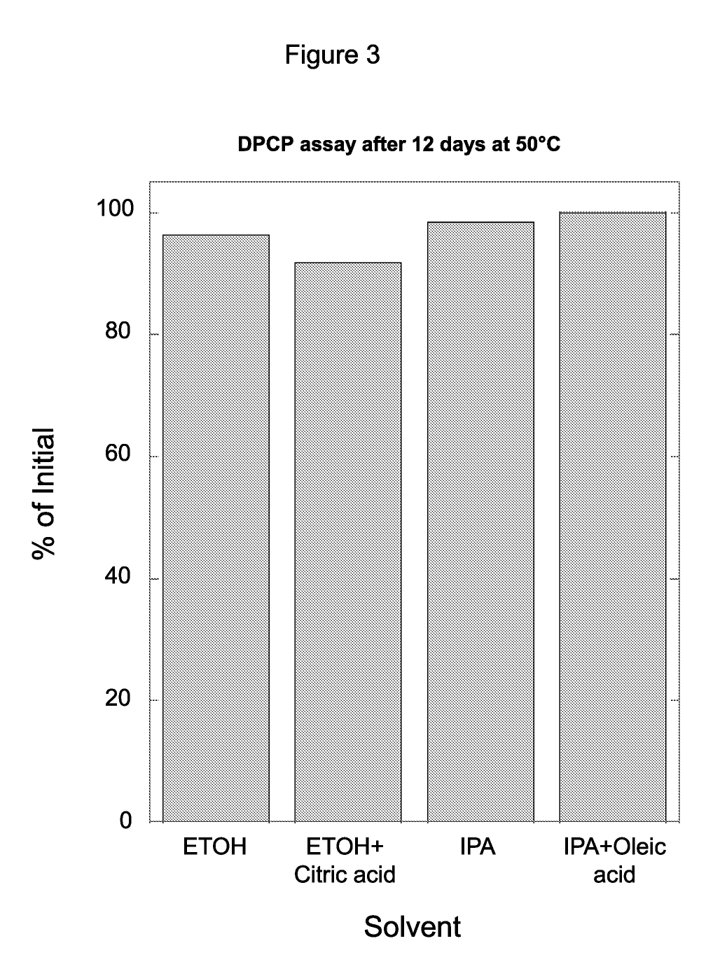

For the development of a gel, the stability of DPCP was determined in both Ethanol (ETOH) and Isopropanol (IPA) was determined (0.4% DPCP solutions in each solvent was placed at 50° C. for two weeks; FIG. 3. All solutions contained 0.1% BHT). Some degradation of DPCP in ETOH was observed in the presence of citric acid. However, DPCP was stable in IPA. The stability of DPCP in the above solvents was determined using reverse phase HPLC on a C18 column.

### Example 10: Ointment Formulations (Ointment 1)

Ointments containing haptens can comprise one or more of the following excipients:

| Excipient | % w/w |
|---|---|
| BHT | 0.1% |
| Methylparaben | 0.1% |
| Propylparaben | 0.05% |
| Cetyl esters wax | 10% |
| White wax | 10% |
| Polysorbate 80^{a} | 39.875% |
| Isopropyl myristate^{a} | 39.875% |

^{a}These excipients can be reduced slightly in formulations containing DPCP

### Example 11: Further Ointment Formulations (Ointment 2)

Ointments containing haptens can comprise one or more of the following excipients:

| Excipient | % w/w |
|---|---|
| BHT | 0.1% |
| Methylparaben | 0.1% |
| Propylparaben | 0.05% |
| Glyceryl monostearate, EP | 5% |
| Cetyl esters wax | 7.5% |
| White wax | 7.5% |
| Polysorbate 80^{a} | 39.875% |
| Isopropyl myristate^{a} | 39.875% |

^{a}These excipients can be reduced slightly in formulations containing DPCP

### Example 12: Gel Formulations

Gels containing haptens can comprise one or more of the following excipients:

| Excipient | % w/w |
|---|---|
| BHT | 0.1% |
| Klucel MF Pharm | 2% |
| Isopropyl alcohol | 57.9% |
| Propylene glycol | 10% |
| Polysorbate 80^{a} | 15% |
| Isopropyl myristate^{a} | 15% |

^{a}These excipients can be reduced slightly in formulations containing DPCP

### Example 13: Stability of Formulations at 3 Weeks

The gel and ointment formulations outlined in Examples 10 to 12 (containing 0.4% DPCP), were manufactured and their stability was monitored over a period of 3 weeks at both 25° C. and 30° C. The appearance, strength of DPCP and viscosity of the formulations were observed. At the 3 week time point, no significant changes were observed at the 25° C. or 30° C. conditions.

TABLE 1
Initial Test Results

| | | Assay, | |
|---|---|---|---|
| Formulation | Appearance | % w/w | Viscosity, cP |
| Ointment 1 (0.4% DPCP) | Off-white to beige | 0.386 | 23,000¹ |
| | homogeneous ointment | | |
| Ointment 2 (0.4% DPCP) | Off-white to beige | 0.392 | 24,500¹ |
| | homogeneous ointment | | |
| Gel (0.4% DPCP) | Clear to translucent | 0.388 | 89,000² |
| | slightly granular gel | | |

¹Rheosys cone/plate, 1 rpm, 20° C.
²Brookfield LV, spindle #14, sample holder #6R, 20° C.

TABLE 2
Assay and Viscosity after 3 weeks at 30° C.:

| | | Assay, | Viscosity, |
|---|---|---|---|
| Formulation | Appearance | % initial | cP |
| Ointment 1 (0.4% DPCP) | Very slightly softened | 99.2 | 22,200¹ |
| | with no syneresis | | |
| Ointment 2 (0.4% DPCP) | Slightly softened with | 98.7 | 24,000¹ |
| | no syneresis | | |
| Gel (0.4% DPCP) | Clear to translucent | 101.2 | 91,000² |
| | slightly granular gel | | |

¹Rheosys cone/plate, 1 rpm, 20° C.
²Brookfield LV, spindle #14, sample holder #6R, 20° C.

TABLE 3
Appearance after 3 weeks:

| Formulation | 25° C. | 30° C. | 40° C. |
|---|---|---|---|
| Ointment 1 (0.4% DPCP) | Off-white to beige | Very slightly | Very soft, |
| | homogeneous | softened with no | pourable with no |
| | ointment | syneresis | syneresis |
| Ointment 2 (0.4% DPCP) | Off-white to beige | Slightly softened | Liquefied, with |
| | homogeneous | with no syneresis | some very slight |
| | ointment | | syneresis |
| Gel (0.4% DPCP) | Clear to translucent | Clear to translucent | Clear to |
| | slightly granular gel | slightly granular gel | translucent slightly |
| | | | granular gel |

In any of the embodiments discussed herein, a hapten, such as DPCP can be topically administered as a gel, ointment or cream. A sensitization dose (in the range of 0.1% DPCP to 1% DPCP) can be provided approximately 2 weeks prior to challenge dose. A challenge dose (in the range of 0.0000001% to 0.4% DPCP) can be provided approximately two weeks post sensitization dose and then approximately twice every week, once every week, once every two weeks or once every three weeks. In case of a relapse, dosing can be re-initiated.

### Example 14: Delayed-Type Hypersensitivity (DTH) Response of Subjects Treated with Ointment #1 Containing 0.4% DPCP Vs Vehicle Ointment (Ointment #1, 0% DPCP)

Table 4 depicts data from a preliminary analysis of the Immunotherapeutic Skin Reaction Scores (scale from 0 to 4) during the Sensitization Phase for a clinical trial evaluating DPCP Ointment for the treatment of warts. Subjects were first treated with Vehicle Ointment #1 and their Immunotherapeutic Skin Reaction was scored. Following the vehicle treatment, subjects received 0.1 mL of Ointment #1 containing 0.4% DPCP on their inner right arm and their Immunotherapeutic Skin reaction was scored 2-14 days later. A score of ≥2 signifies the subject was sensitized to the treatment. Subjects did not demonstrate

What is claimed is:

1. A method for treating cutaneous neoplasm, the method comprising administering to a subject in need thereof a therapeutically effective amount of a hapten that elicits a T-cell response.

2. The method of claim 1, wherein the cutaneous neoplasm is selected from the group consisting of basal cell carcinoma, squamous cell carcinoma, bowen's disease (pre-invasive squamous cell carcinoma), actinic keratosis, metastatic merkel cell carcinoma, and cutaneous T-cell lymphoma.

3. The method of claim 1 or 2, wherein the hapten is selected from the group consisting of diphenylcyclopropenone (DPCP), imiquimod, ingenol mebutate, and Squaric Acid Dibutylester (SADBE).

4. The method of claim 3, wherein the hapten is DPCP.

5. A method for the treatment of cutaneous metastasis in a subject having solid neoplasia, the method comprising administering to a subject in need thereof a therapeutically effective amount of a hapten that elicits a T-cell response.

6. The method of claim 5, wherein the hapten is administered as an adjunct or adjuvant therapy.

7. The method of claim 6, wherein the subject is also receiving one or more anti-neoplastic therapies and/or one or more radiation therapies.

8. The method of any of claims 5-7, wherein the cutaneous metastasis originates from a solid neoplasia selected from the group consisting of breast carcinoma, lung carcinoma, bladder carcinoma, colon carcinoma, uterine carcinoma, renal carcinoma, gastrointestinal carcinoma, and oral squamous cell carcinoma.

9. The method of claim 8, wherein the cutaneous metastasis is located in one or more cutaneous areas in a subject selected from the group consisting of the chest, abdomen, lower abdomen, back, scalp, head, neck, leg, arm, and other extremities, genitalia, pubic area, and pelvis areas.

10. The method of any of claims 5-9, wherein the hapten is selected from DPCP, imiquimod, ingenol mebutate, and SADBE.

11. The method of claim 10, wherein the hapten is DPCP.

12. A method for the complete or partial removal of a skin tattoo, the method comprising administering to a subject in need thereof a therapeutically effective amount of a hapten that elicits a T-cell response.

13. The method of claim 12, wherein the hapten is administered as an adjunct or adjuvant therapy.

14. The method of claim 13, wherein the subject is also receiving laser treatment to completely or partially remove the tattoo.

15. The method of any of claims 12-14, wherein the hapten is selected from DPCP, imiquimod, ingenol mebutate, and SADBE.

16. The method of claim 15, wherein the hapten is DPCP.

17. The method of any of claims 1 to 16, wherein the hapten is administered to the skin of the subject as an initial sensitizing dose and a subsequent challenge dose.

18. The method of claim 17, wherein said initial sensitizing dose and said subsequent challenge dose are administered to the same site on the skin.

19. The method of claim 18, wherein said initial sensitizing dose is administered to a first site on the skin and said subsequent challenge dose is administered to second site on the skin that differs from the first site.

20. The method of any of claims 1 to 19, wherein the hapten is formulated in a composition comprising a gel formulation.

21. The method of claim 20, wherein the composition comprises (a) a non-ionic surfactant, (b) an alcoholic ester, and (c) a gelling agent.

22. The method of claim 21, wherein said non-ionic surfactant is selected from the group consisting of polyoxyethylene (20) monoleate, polyoxyethylene (20) sorbitan monooleate, polysorbate 80, palmitate, and stearate.

23. The method of claim 21 or 22, wherein said alcoholic ester is selected from the group consisting of isopropyl myristate and isopropyl palmitate.

24. The method of any of claims 21-23, wherein said gelling agent is selected from the group consisting of polyoxyl 40 stearate and hydroxypropyl cellulose.

25. The method of any of claims 21-24, wherein the gel composition comprises polysorbate 80, isopropyl myristate, and polyoxyl 40 stearate.

26. The method of any of claims 20-25, wherein the gel composition comprises from about 0.1% to about 1% hapten.

27. The method of claim 26, wherein the gel composition comprises 0.4% hapten.

28. The method of any of claims 20-25, wherein the gel composition comprises from about 0.0000001% to about 0.4% hapten.

29. The method of any of claims 17-28, wherein the first challenge dose of hapten is administered two weeks or about two weeks subsequent to the administration of the sensitizing dose of hapten.

30. The method of any of claims 17-29, wherein said challenge dose is administered to the skin daily.

31. The method of any of claims 17-29, wherein said challenge dose is administered to the skin every other day.

32. The method of any of claims 17-29, wherein said challenge dose is administered to the skin twice a week.

33. The method of any of claims 17-29, wherein said challenge dose is administered to the skin weekly.

34. The method of any of claims 17-29, wherein said challenge dose is administered to the skin every two weeks.

35. The method of any of claims 17-29, wherein said challenge dose is administered to the skin every three weeks.

36. The method of any of claims 17-29, wherein said challenge dose is administered to the skin monthly.

37. The method of any of claims 17-29, wherein said challenge dose is administered to the skin in any combination of daily, every other day, twice a week, weekly, every other week, every three weeks and/or monthly administration.

38. The method of any of claims 17-37, wherein the hapten is selected from DPCP, imiquimod, ingenol mebutate, and SADBE.

39. The method of claim 38, wherein the hapten is DPCP.

40. A composition for use in the treatment of a cutaneous neoplasm in a subject, wherein the composition comprises a hapten that elicits a T-cell response, a non-ionic surfactant, an alcoholic ester, and a gelling agent.

41. A composition for use in the treatment of cutaneous metastasis in a subject having solid neoplasia, wherein the composition comprises a hapten that elicits a T-cell response, a non-ionic surfactant, an alcoholic ester, and a gelling agent.

42. A composition for use in the complete or partial removal of a skin tattoo on a subject, wherein the composition comprises a hapten that elicits a T-cell response, a non-ionic surfactant, an alcoholic ester, and a gelling agent.

43. The composition of any of claims 40-42, wherein the non-ionic surfactant is selected from the group consisting of polyoxyethylene (20) monoleate, polyoxyethylene (20) sorbitan monooleate, polysorbate 80, palmitate, and stearate.

44. The compositions of any of claims 40-42, wherein the alcoholic ester is selected from is selected from the group consisting of isopropyl myristate and isopropyl palmitate.

45. The compositions of any of claims 40-42, wherein the gelling agent is selected from the group consisting of polyoxyl 40 stearate and hydroxypropyl cellulose.

46. The composition of any of claims 40-45, wherein the hapten is selected from DPCP, imiquimod, ingenol mebutate, and SADBE.

47. The composition of claim 46, wherein the hapten is DPCP.

48. The composition of claim 47 comprising DPCP, Polysorbate 80, Isopropyl myristate, and Polyoxyl 40 Stearate.

49. A formulation for use in the treatment of a cutaneous neoplasm in a subject, comprising DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

50. A formulation for use in the treatment of cutaneous metastasis in a subject having solid neoplasia, comprising DPCP, 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

51. A formulation for use in the complete or partial removal of a skin tattoo on a subject, comprising 0.02% Butylated hydroxytoloune (BHT), 43.4125-43.915% Polysorbate 80, 43.4125-43.915% Isopropyl myristate, 12% Polyoxyl 40 Stearate, 0.1% Methyl Paraben and 0.05% Propyl Paraben.

52. The formulation of any of claims 49-51 comprising from about 0.1% to about 1% DPCP.

53. The formulation of any of claims 49-51 comprising 0.4% DPCP.

54. The formulation of any of claims 49-51 comprising from about 0.0000001% to about 0.4% DPCP.

55. A method for the treatment of cutaneous neoplasm in a subject, the method comprising (a) administering to the skin of said subject a sensitizing dose of hapten; (b) administering to the skin of said subject a first challenge dose of hapten; and (c) continuing to administer to the skin of said subject one or more further challenge dose(s) of hapten according to a pre-determined schedule until the cutaneous neoplasm has been treated.

56. A method for the treatment of cutaneous metastasis in a subject having solid neoplasia, the method comprising (a) administering to the skin of said subject a sensitizing dose of hapten; (b) administering to the skin of said subject a first challenge dose of hapten; and (c) continuing to administer to the skin of said subject one or more further challenge dose(s) of hapten according to a pre-determined schedule until the cutaneous metastasis has been treated.

57. A method for the complete or partial removal of a tattoo in a subject, the method comprising (a) administering to the skin of said subject a sensitizing dose of hapten; (b) administering to the skin of said subject a first challenge dose of hapten; and (c) continuing to administer to the skin of said subject one or more further challenge dose(s) of hapten according to a pre-determined schedule until the tattoo has been completely or partially removed.

58. A method for treating a wart, the method comprising administering to a subject in need thereof a therapeutically effective amount of a hapten that elicits a T-cell response.

59. A method for treating cutaneous metastasis of malignant melanoma, the method comprising administering to a subject in need thereof a therapeutically effective amount of a hapten that elicits a T-cell response.

60. The method of claim 58 or 59, wherein the hapten is selected from the group consisting of diphenylcyclopropenone (DPCP), imiquimod, ingenol mebutate, and Squaric Acid Dibutylester (SADBE).

61. The method of claim 60, wherein the hapten is DPCP.

62. The method of any of claims 58-61, wherein the hapten is formulated in a composition comprising a gel formulation.

63. The method of claim 62, wherein the composition comprises (a) a non-ionic surfactant, (b) an alcoholic ester, and (c) a gelling agent.

64. The method of claim 63, wherein said non-ionic surfactant is selected from the group consisting of polyoxyethylene (20) monoleate, polyoxyethylene (20) sorbitan monooleate, polysorbate 80, palmitate, and stearate.

65. The method of claim 63 or 64, wherein said alcoholic ester is selected from the group consisting of isopropyl myristate and isopropyl palmitate.

66. The method of any of claims 63-65, wherein said gelling agent is selected from the group consisting of polyoxyl 40 stearate and hydroxypropyl cellulose.

67. The method of any of claims 62-66, wherein the composition comprises polysorbate 80, isopropyl myristate, and polyoxyl 40 stearate.

68. The method of any of claims 62-67, wherein the composition comprises from about 0.1% to about 1% hapten.

69. The method of claim 68, wherein the composition comprises 0.4% hapten.

70. The method of any of claims 62-69, wherein the composition comprises from about 0.0000001% to about 0.4% hapten.

71. A composition for use in the treatment of a wart in a subject, wherein the composition comprises a hapten.

72. A composition for use in the treatment of cutaneous metastasis of malignant melanoma in a subject, wherein the composition comprises a hapten.

73. The composition of claim 71 or 72, wherein the hapten is selected from DPCP, imiquimod, ingenol mebutate, and SADBE.

74. The composition of claim 73, wherein the hapten is DPCP.

75. A method for the treatment of a wart in a subject, the method comprising (a) administering to the skin of said subject a sensitizing dose of hapten; (b) administering to the skin of said subject a first challenge dose of hapten; and (c) continuing to administer to the skin of said subject one or more further challenge dose(s) of hapten according to a pre-determined schedule until the wart has been treated.

76. A method for the treatment of cutaneous metastasis of malignant melanoma in a subject, the method comprising (a) administering to the skin of said subject a sensitizing dose of hapten; (b) administering to the skin of said subject a first challenge dose of hapten; and (c) continuing to administer to the skin of said subject one or more further challenge dose(s) of hapten according to a pre-determined schedule until the cutaneous metastasis of malignant melanoma has been treated.

77. The method of claim 75 or 76, wherein the hapten is formulated in a composition comprising a gel formulation or an ointment formulation.

78. A composition comprising a hapten gel formulation, wherein the composition comprises a) a first co-solvent comprising a non-ionic surfactant, b) a second co-solvent comprising an alcoholic ester, and c) a gelling agent.

79. The composition of claim 78, wherein said first co-solvent is selected from the group consisting of polyoxyethylene (20) monoleate, polyoxyethylene (20) sorbitan monooleate, polysorbate 80, palmitate and stearate, and wherein said second co-solvent is selected from the group consisting of isopropyl myristate and isopropyl palmitate, and wherein said gelling agent is selected from the group consisting of polyoxyl 40 stearate and hydroxypropyl cellulose.

80. The composition of claim 79, wherein the composition comprises 0.01 to 1% BHT, 10 to 20% Polysorbate 80, 10 to 20% Isopropyl myristate, 5 to 15% Propylene glycol, 0.1 to 5% Klucel and 40 to 70% Isopropyl alcohol.

81. The composition of any one of claims 78-80, wherein the hapten is DPCP, imiquimod, ingenol mebutate or SADBE.

82. The composition of claim 81, wherein the hapten is DPCP.

83. A composition comprising a hapten ointment formulation, wherein the composition comprises a) a first co-solvent comprising a non-ionic surfactant, b) a second co-solvent comprising an alcoholic ester, and c) a thickening agent.

84. The composition of claim 83, wherein said first co-solvent is selected from the group consisting of polyoxyethylene (20) monoleate, polyoxyethylene (20) sorbitan monooleate, polysorbate 80, palmitate and stearate, wherein said second co-solvent is selected from the group consisting of isopropyl myristate and isopropyl palmitate, and wherein said thickening agent is selected from the group consisting of white wax, cetyl ester wax and glyceryl monosterate.

85. A composition comprising a hapten ointment formulation, wherein the composition comprises 0.01 to 1% BHT, 20 to 50% Polysorbate 80, 20 to 50% Isopropyl myristate, 2.5 to 20% White wax, 2.5 to 20% Cetyl esters wax, 0 to 10% glyceryl monostearate, 0 to 1% methylparaben and/or 0 to 1% propylparaben.

86. The composition of any one of claims 83-85, wherein the hapten is DPCP, imiquimod, ingenol mebutate or SADBE.

87. The composition of claim 86, wherein the hapten is DPCP.

88. The composition of claim 87, wherein the dose of DPCP is 0.0000001% to about 1%.

89. A method comprising administering to a subject in need thereof a therapeutically effective amount of a composition comprising a hapten gel formulation, wherein the composition comprises a) a first co-solvent comprising a non-ionic surfactant, b) a second co-solvent comprising an alcoholic ester, and c) a gelling agent.

90. The method of claim 89, wherein said first co-solvent is selected from the group consisting of polyoxyethylene (20) monoleate, polyoxyethylene (20) sorbitan monooleate, polysorbate 80, palmitate and stearate, and wherein said second co-solvent is selected from the group consisting of isopropyl myristate and isopropyl palmitate, and wherein said gelling agent is selected from the group consisting of polyoxyl 40 stearate and hydroxypropyl cellulose.

91. The method of claim 90, wherein the gel composition is comprised of 0.01 to 1% BHT, 10 to 20% Polysorbate 80, 10 to 20% Isopropyl myristate, 5 to 15% Propylene glycol, 0.1 to 5% Klucel and 40 to 70% Isopropyl alcohol.

92. The method of any one of claims 89-91, wherein the hapten is DPCP, imiquimod, ingenol mebutate or SADBE.

93. The method of claim 92, wherein the hapten is DPCP.

94. A method comprising administering to a subject in need thereof a therapeutically effective amount of a composition comprising a hapten ointment formulation, wherein the composition comprises a) a first co-solvent comprising a non-ionic surfactant, b) a second co-solvent comprising an alcoholic ester, and c) a thickening agent.

95. The method of claim 94, wherein said first co-solvent is selected from the group comprising polyoxyethylene (20) monoleate, polyoxyethylene (20) sorbitan monooleate, polysorbate 80, palmitate and stearate, and wherein said second co-solvent is selected from the group comprising of isopropyl myristate and isopropyl palmitate, and wherein said thickening agent is selected from the group comprising of white wax, cetyl ester wax and glyceryl monosterate.

96. A method comprising administering to a subject in need thereof a therapeutically effective amount of a composition comprising a hapten ointment formulation, wherein the ointment is comprised of 0.01 to 1% BHT, 20 to 50% Polysorbate 80, 20 to 50% Isopropyl myristate, 2.5 to 20% White wax, 2.5 to 20% Cetyl esters wax, 0 to 10% glyceryl monostearate, 0 to 1% methylparaben and/or 0 to 1% propylparaben.

97. The method of any one of claims 94-96, wherein the hapten is DPCP, imiquimod, ingenol mebutate or SADBE.

98. The method of claim 97, wherein the hapten is DPCP.

99. The method of any one of claims 89-98, wherein the hapten is DPCP and wherein the dose of DPCP is about 0.0000001% to about 1%.

100. A method comprising administering a therapeutically effective amount of a composition of claims 78-88 to a subject in need thereof.

101. The method of claim 100, wherein the method is a method for treating cutaneous neoplasm, cutaneous metastasis in a subject having solid neoplasia, cutaneous metastasis of malignant melanoma, or a wart, or wherein the method is a method for tattoo removal.

102. The method of claims 89-98, wherein a low sensitizing dose of said composition is administered to a first site on the skin of a human patient followed by a subsequent administration to a second site on the skin of said patient a challenge dose of said composition, wherein said composition comprises DPCP.

103. The method of claim 102, wherein said low sensitizing dose is about 0.1 to about 1% DPCP, and wherein the challenge dose is 0.0000001% to about 0.4% DPCP.

104. The method of claim 103, wherein said sensitizing dose is 0.4% DPCP.

105. The method of claim 102, wherein said challenge dose is administered to the skin daily.

106. The method of claim 102, wherein said challenge dose is administered to the skin every other day.

107. The method of claim 102, wherein said challenge dose is administered to the skin twice a week.

108. The method of claim 102, wherein said challenge dose is administered to the skin weekly.

109. The method of claim 102, wherein said challenge dose is administered to the skin every two weeks.

110. The method of claim 102, wherein said challenge dose is administered to the skin every three weeks.

111. The method of claim 102, wherein said challenge dose is administered to the skin in any combination of daily, every other day, twice a week, weekly, every other week, every three weeks and/or monthly.
